(19) United States
(12) Patent Application Publication
(10) Pub. No.: US 2009/0097026 A1
(43) Pub. Date: Apr. 16, 2009

(54) COLOR MEASUREMENTS OF RECONSTITUTED TIGECYCLINE AND DEGRADANT THEREOF

(75) Inventors: Jean Marie Akin, Cross River, NY (US); Peter John Larkin, Trumbull, CT (US); Tianmin Zhu, Monroe, NY (US); Jan Szeliga, Croton on Hudson, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/247,269

(22) Filed: Oct. 8, 2008

Related U.S. Application Data
(60) Provisional application No. 60/998,634, filed on Oct. 12, 2007.

(51) Int. Cl. G01N 21/31; C07C 237/28

(52) U.S. Cl. 356/319; 564/157

(57) ABSTRACT

The present invention relates to a color method and multivariate model whose color measurements of reconstituted tigecycline for injection is predictive of appearance and oxidative degradation wherein said method is a means of detecting the presence of a degradant which forms when tigecycline is contacted with oxygen.

### CROSS-REFERENCE TO RELATED APPLICATIONS

This application claims the benefit under 35 U.S.C. §119(e) to co-pending U.S. Provisional Application Ser. No. 60/998,634, filed Oct. 12, 2007, which is hereby incorporated by reference in its entirety.

### FIELD OF THE INVENTION

The present invention relates to a color method and multivariate model whose color measurements of reconstituted tigecycline for injection is predictive of appearance and oxidative degradation.

The rapid emergence of antimicrobial drug resistance has been recognized as an epidemic of global proportions. (O'Brien T F. 1997. The Global epidemic nature of antimicrobial resistance and the need to monitor and manage it locally. Clin Infect Dis 24(Suppl 1): S2-8) Particularly in hospital-acquired infections, many pathogenic bacteria are multiply resistant to several classes of antibiotics, effectively narrowing therapeutic options. (White D G, McDermott P F. 2001. Emergence and transfer of antibacterial resistance. J Dairy Sci 84(7):E151-E155.) For multiply resistant Staphylococcus aureus, vancomycin currently represents the last line of defense. Recent reports of S. aureus lacking susceptibility to vancomycin underscore the looming threat of untreatable infections. (Denis O, Deplano A, Nonhoff C, Hallin M, De Ryck R, Vanhoof R, De Mendonca R, Struelens M J. 2006. In vitro activities of ceftobiprole, tigecycline, daptomycin, and 19 other antimicrobials against methicillin-resistant Staphylococcus aureus strains from a national survey of Belgian hospitals. Antimicrob Agents Chemother 50(8):2680-2685.) In 2005, tigecycline, a novel intravenous antibiotic with a broad spectrum of antimicrobial activity, including activity against drug-resistant bacteria such as methicillin-resistant Staphylococcus aureus was approved and launched. (Sum, P-E. 2006. Case studies in current drug development: ‘glycylcyclines’. Curr Opin Chem Biol 10(4):374-379.) Tygacil has proven efficacious in clinical trials for the treatment of complicated skin and skin structure infections and complicated intra-abdominal infections in adults. (Van Wart S A, Owen J S, Ludwig E A, Meagher A K, Korth-Bradley J M, Cirincione B B. 2006. Population pharmacokinetics of tigecycline in patients with complicated intra-abdominal or skin and skin structure infections. Antimicrob Agents Chemother 50(11):3701-3707.) This first-in-class glycylcycline product has provided physicians with an important alternative option to overcome the problems of resistance observed with the other antibiotics and to combat serious, resistant infections for all patients.

Tigecycline for Injection is supplied as a sterile, lyophilized yellow/orange powder for reconstituted intravenous infusion in single dose Type 1 glass vials (50 mg/vial) and does not contain excipients or preservatives. Each vial is sealed with a rubber stopper. However, tigecycline has been found to be susceptible to oxidative degradation and each supplied vial is stored under a blanket of nitrogen. Each tigecycline vial contains 50 mg of tigecycline powder which is reconstituted with 5 mL normal saline or 5% dextrose solution and immediately diluted into an IV bag at a final concentration of 0.5 mg/mL for intravenous infusion.

When the lyophilized tigecycline powder is reconstituted the resulting intravenous solution slowly begins to turn from yellow/orange to dark green. While the reconstituted solution is rapidly administered to patients in need thereof, there is a further need in the art to determine a color method and multivariate model whose color measurements of reconstituted tigecycline are predictive of appearance and oxidative degradation.

Color measurements have been used extensively to quantify product appearance in industries such as coatings, plastics and food. This established technology has been less frequently utilized in the pharmaceutical industry. Instrumental color measurements are used to obtain repeatable numeric values that correspond to visual assessment. The color spectrophotometer uses the entire visible spectrum of light that is transmitted through, or reflected from a sample. Mathematical tables representative of the human eye's color sensitivity and the color output of different light sources are used to calculate color indices. The well-known CIE Commission International de l'Elclairage (International Commission on Illumination) L*a*b* color scale provides a three dimensional, linear, color scale that is organized in a cube form. The present invention demonstrates the value of applying color measurements as a diagnostic of product appearance for the intravenous drug product tigecycline.

### BRIEF DESCRIPTION OF THE DRAWINGS

FIG. 1 shows the proton nuclear magnetic resonance spectrum of Compound I

FIG. 2 shows the UV-VIS spectrum of tigecycline and Compound I

### SUMMARY OF THE INVENTION

In accordance with the invention, a method of detecting and measuring degradant formation in tigecycline is provided.

The invention provides a method of detecting in tigecycline the presence of the degradant, Compound I, having the structure:

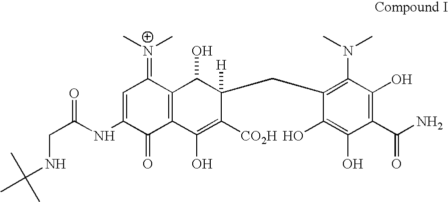

In further embodiment of the invention there is provided a method of measuring the color of tigecycline wherein the color value is determined by the equation: Calculated color value=(2.19×L*+2.59×a*−0.48×b*) where color indices L*, a* and b* are measured spectophotometrically. The present invention also provides a method for determining the acceptability of a sample of Tygecycline with respect to degradation to Compound I.

The invention further provides substantially pure Compound I.

The invention further provides a method of detecting in tigecycline the presence of a compound containing the structure:

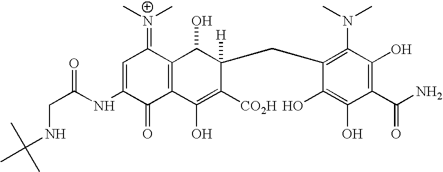

and the anion of a pharmaceutically acceptable salt.

The invention further provides a substantially pure compound containing the structure:

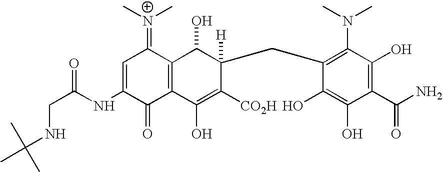

and the anion of a pharmaceutically acceptable salt.

### DEFINITIONS

As used herein TOCSY means total correlation spectroscopy.

As used herein HSQC means heteronuclear single quantum correlation spectroscopy.

As used herein HMBC means heteronuclear multiple bond coherence spectroscopy.

As used herein MS means mass spectroscopy.

As used herein LC-MS means liquid chromatography coupled mass spectroscopy.

As used herein LC-MS/MS means liquid chromatography tandem mass spectroscopy.

As used herein HPLC means high pressure liquid chromatography.

As used herein NMR means nuclear magnetic resonance spectroscopy.

As used herein, the term “substantially pure” means a purity of about 90% by weight or greater.

As used herein, the term “pharmaceutically acceptable salt”, as used herein, refers to salts derived form organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids.

### DETAILED DESCRIPTION OF THE INVENTION

Tigecycline for Injection (50 mg/vial) is a sterile, lyophilized powder for intravenous infusion. The product is typically supplied in single dose Type 1 glass vials, each sealed with a rubber stopper under a blanket of nitrogen. The color methodology of this invention is based upon color measurements of oxygen-stressed reconstituted tigecycline solutions and enabled detectable degradant color formation. In one embodiment, the reconstituted tigecycline solution is measured in transmittance mode using a fixed path length cell (10 mm) on a HunterLab UltraScana XE Color Spectrophotometer. Transmittance as defined in the present invention is the process by which incident light is transmitted through a solution of tigecycline. Based upon the measured CIE indices of L*, a*, b*, the calculated color value is determined using the following developed discriminator equation: calculated color value=2.19×L*+2.59×a*−0.48×b*.

The resultant color method and multivariate model provides a simple, robust and quantitative predictive tool of tigecycline color performance. The acceptance criteria provides a simple, predictive benchmark of the product appearance and is based upon oxidative degradation of the tigecycline moiety.

The discriminator was built using the minimum Expected Cost of Misclassification Rule resulting in an equation of the form: (k1*L+k2*a+k3*b). The calculated color values and acceptability criterion are based upon a statistical analysis of ten lots of tigecycline for injection 50 mg/vial. The model was derived by equating the ratio of the two types of misclassifications to the ratio of the population proportion of unacceptable lots to acceptable ones. It has been discovered that when k1 is approximately 2.19, k2 is approximately 2.59, and k3 is approximately −0.48, the equation is a very useful tool for determining the degree to which a sample of Tygecycline has degraded to Compound I.

In one embodiment of the method of the present invention, a color spectrometer is used to measure the CIE indices L*, a*, and b* of a sample of tigecycline by procedures well-known in the art. The measured values of L*, a*, and b* are then inserted into the equation: calculated color value=2.19×L*+2.59×a*−0.48×b*. The calculated color value indicates the degree of degradation of the tygecycline to Compound I; a lower value indicates a greater degree of degradation. In a further embodiment of the method of this invention, the calculated color value is compared to 150 and the sample is determined to be unacceptable if the value is less than 150.

In the practice of this invention, the color value measurements of tigecycline samples may be made at a single point in time, at multiple points in time, or continuously to monitor changes over time. The choice of color measurement protocols will depend on the equipment available and the purpose of the measurements. For example, a single measurement may be made prior to shipping the product to ensure product quality. Multiple or continuous measurements of a sample may be needed where changes over time are being studied.

The invention further provides the discovery of a tigecycline degradation product having the structure of Compound I, having an iminoquinone moiety and a unique intense blue color.

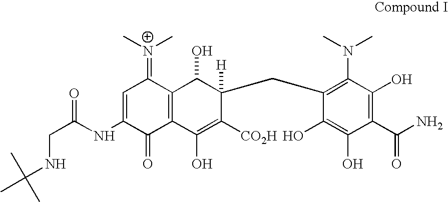

Compound I was isolated from forced degradation samples of tigecycline and is unstable. This blue compound was characterized by LC-MS/MS and NMR as an iminoquinone derivative of tigecycline, an oxidative degradant with a positive ionic formula of C₂₉H₃₈N₅O₁₀ formed when tigecycline is exposed to air for twenty-four hours. A linear correlation (R>0.99) between the concentration of Compound I and the calculated color value was discovered. In substantially pure form, Compound I is useful as a test standard for determining the concentration of Compound I in a sample of Tigecycline. Compound I may be obtained in substantially pure form from the compound Tygecycline according to the following method: Tygecycline is degraded at room temperature by dissolving it in water open to air and stirring for a few hours until the solution changes color from yellow/orange to dark green. The degradation mixture may then be separated into component compounds by any method known in the art, for example by HPLC, to obtain substantially pure Compound I. Other methods of preparing and/or isolating Compound I may be apparent to those skilled in the art from the description and examples provided herein and from common knowledge in the art.

### EXAMPLES

The following Examples are provided to illustrate the invention in greater detail, and are not to limit the scope of the claims of the invention.

### Experimental 1

The inherent time dependent degradation of reconstituted tigecycline, lyophilized 50 mg/vial in particular, requires the use of a simulated sample for accuracy, precision and specificity. The accuracy testing was performed and involved measurements of United States Pharmacopeia (USP) color reference solution standards. Likewise, specificity and repeatability precision testing were also performed on these USP solution reference standards.

All transmission measurements of colored solutions were made using a HunterLab UltraScan XE color spectrophotometer using a 10 mm pathlength cell, D65/10′, 0.375 size and TTRAN) which is a combination of regular (measurement made with sample situated at lens with only transmitted light passing straight through the sample) and diffuse (light scattered through the lens) transmission.

The test method to evaluate the batch-to-batch consistency of oxygen-stressed reconstituted tigecycline directs the analyst to reconstitute tigecycline samples with a defined period of oxygen exposure. The samples are then measured in transmittance mode using a fixed path length cell (10 mm) on a HunterLab UltraScan XE Color Spectrophotometer. Based upon the measured CIE indices of L*, a*, b*, a calculation using the following discriminator equation is derived that determines whether or not a tigecycline lot is of good color quality:

Calculated color value: 2.19×L*+2.59×a*−0.48×b*<150.

The acceptance criterion was defined that a tigecycline lot would be determined to have unacceptable color if the calculated color value was below 150.

Accuracy: Three different color reference solutions: J, L and O were measured. Each reference solution was measured 3 times (N=3) (Fluka, Colour Reference Solutions acc. To USP, 2 ml, product no. 87576). Samples were transferred from a glass ampoule and measured using a 0.5 cm pathlength cell.

Specificity: Compared CIE L*a*b* color indices of color reference solutions (Fluka, Colour Reference Solutions acc. To USP, 2 ml, product no. 87576). Five individual standards (F, J, L, and Q) were measured that vary in color to demonstrate the variance in color CIE L*A*B* indices. Measurements were made 3 times (N=3). Samples were transferred from a glass ampoule and measured using a 0.5 cm pathlength cell.

Precision (repeatability): Fluka color reference solution “L” (Fluka, Colour Reference Solutions acc. To USP, 2 ml, product no. 87576) was measured six (N=6) times Samples were transferred from a glass ampoule and measured in a 0.5 cm pathlength cell.

Precision (intermediate): Twelve (N=12) measurements of three batches of tigecycline batches. Batches to include Patheon TR001 and Carolina 4990-01-B41133 of tigecycline lactose formulation 50 mg/vial, as well as one batch (A93983) of tigecycline for injection 50 mg/vial. Three vials from each batch were tested on each of two days.

Robustness here is defined as changes made in experimental parameters in order to determine factors causing test result variability. The color test is defined as the characterization of the color of solution using a color spectrophotometer with both the conditions and the acceptance criteria defined by the test method; using Batch #4990-01-B41133 (tigecycline for injection 50 mg/vial, lactose formulation) and A93983 (tigecycline for injection 50 mg/vial) which will include the variables of measurement of time on reconstituted tigecycline (3.5 min) and saline solution volume (5.3 ml and ±0.1 ml), 2 vials each batch each test.

System Suitability As per blank standardization and green tile test. Using a green tile with predetermined vendor values, make color measurement at midrange to demonstrate instrument is working properly.

Acceptance Criteria—L*a*b* values and for tigecycline is the calculated color values defined by the method described herein. Criteria are defined by the experimental protocol set for accuracy, precision, robustness, and specificity.

Accuracy: The method accurately distinguishes the intensity of color among the three standards, which are representative of the expected sample color range. The mean L values obtained for three different color reference solutions: J, L and O differed from one another by more than 5 units. The mean L values obtained for three different color reference solutions: J, L, O differed from one another by more than 5 units. The limit of 5 units was selected as an approximation of visual discrimination by a typical observer for color standards with variations in the degree of perceived yellowness. This demonstrates that the method accurately distinguishes the intensity of color among the three standards, which are representative of the expected sample color range.

Precision (repeatability): CIE L*A*B* values, mean and standard deviation for L Fluka color reference solutions and the relative standard deviation (RSD (%) or coefficient of variation (CV) for the L standard was: L*=−0.08, a*=0.49, and b*=0.82.

Accuracy: Precision (repeatability): Reported CIELAB values, mean and standard deviation for L Fluka color reference solutions. The RSD (%) or CV for the L standard was: L*=−0.08, a*=0.49, and b*=0.82.

Precision (intermediate): Each analyst reports the individual and mean CIELAB values for each batch for each day and overall. The overall mean results meet the following:
1. Evaluate the CIELAB results for each batch between the WP and AQS analysts as follows:
   a. The mean values for L* for each analyst agree within 1 unit
   b. The mean values for a* for each analyst agree within 1 unit
   c. The mean values for b* for each analyst agree within 1 unit
2. The mean calculated values for each batch agree within 1 unit between the two analyst.
3. The overall RSD (%) from each laboratory for the calculated values for each batch are not more than 1.0.

Specificity: CIE color indices differentiated among all five (F, J, L, O, Q) Fluka color reference solutions. The a* color indices vary from 0.5 (slightly red) to −16.55 (solidly green). The b* indices vary from 2.67 (light yellow) to 60.58 (dark yellow). The transmittance of all five solutions are similar resulting in only minor differences in the L* indices (94.24 to 98.14). These results demonstrate the specificity of the CIE L*a*b* indices using a color spectrophotometer for colored solutions.

Robustness: Color test was performed using tigecycline for injection 50 mg/vial (A93983) and tigecycline for injection lactose formulation (4990-01-B41133).

Variables include measurement time on reconstituted tigecycline (15 min and ±2 min) and saline solution volume (5.3 ml and ±0.1 ml)

Precision (intermediate): The individual and mean CIE L*A*B* values for each batch for each day and overall are reported and the overall mean results meet the following:

a. The mean values for L* agree within 1 unit
b. The mean values for a* agree within 1 unit
c. The mean values for b* agree within 1 unit
d. The mean calculated values for each batch agree within 2 units.

The overall RSD (%) from each independent analysis for the calculated values for each batch are not more than 1.0.

Specificity: CIE L*A*B* color indices differentiated among all five (F, J, L, O and Q) Fluka color reference solutions. The a* color indices vary from 0.5 (slightly red) to −16.55 (solidly green). The b* indices vary from 2.67 (light yellow) to 60.58 (dark yellow). The transmittance of all five solutions are similar resulting in only minor differences in the L* indices (94.24 to 98.14). These results demonstrate the specificity of the CIE L*a*b* indices using a color spectrophotometer for colored solutions. These are the measured CIEL*a*b* values for color standards.

Specificity Data using Fluka Reference Solutions

| Reference | | | | |
|---|---|---|---|---|
| Standard | Sample # | L* | a* | b* |
| L | 1 | 94.23 | −8.06 | 52.07 |
| | 2 | 94.24 | −8.06 | 51.97 |
| | 3 | 94.25 | −8.07 | 52.00 |
| Mean | | 94.24 | −8.06 | 52.01 |
| F | 1 | 98.14 | −3.75 | 15.78 |
| | 2 | 98.14 | −3.74 | 15.73 |
| | 3 | 98.14 | −3.74 | 15.73 |
| Mean | | 98.14 | −3.74 | 15.75 |
| J | 1 | 96.09 | −11.29 | 45.14 |
| | 2 | 96.10 | −11.29 | 45.14 |
| | 3 | 96.10 | −11.28 | 45.08 |
| Mean | | 96.10 | −11.29 | 45.12 |
| O | 1 | 96.96 | −16.55 | 60.58 |
| | 2 | 96.97 | −16.54 | 60.36 |
| | 3 | 96.99 | −16.53 | 60.18 |
| Mean | | 96.97 | −16.54 | 60.37 |
| Q | 1 | 99.01 | 0.50 | 2.65 |
| | 2 | 99.01 | 0.50 | 2.67 |
| | 3 | 99.01 | 0.49 | 2.68 |
| Mean | | 99.01 | 0.50 | 2.67 |

Robustness: The CIE L*A*B* results for batches: A93983 and B41133 were evaluated for volume variation.

Robustness Data: Variation in Measurement Time and Volume of
Diluent (Batch A93983)

| | | | | Calculated |
|---|---|---|---|---|
| Variable | L* | a* | b* | Color Value |
| 13 minutes | 81.70 | 18.68 | 130.06 | 164.87 |
| 15 minutes | 81.23 | 19.10 | 129.70 | 165.10 |
| 17 minutes | 81.60 | 18.66 | 129.73 | 164.76 |
| 5.2 mL | 81.06 | 19.41 | 130.23 | 165.28 |
| 5.3 mL | 81.43 | 18.72 | 130.36 | 164.24 |
| 5.4 mL | 81.93 | 17.94 | 129.54 | 163.71 |

a. The values for L* agree within 2 units of the mean value (intermediate precision)
b. The values for a* agree within 2 units of the mean value (intermediate precision)

The values for b* agree within 2 units of the mean value (intermediate precision)

The time CIE L*A*B* results agree as follows:

a. The values for L* agree within 2 units of the mean value (intermediate precision)
b. The values for a* agree within 2 units of the mean value (intermediate precision)

The values for b* agree within 2 units of the mean value (intermediate precision)

### Experimental 2

Color Measurements of transparent, colored solutions of tigecycline for Injection (50 mg/vial) to evaluate batch-to-batch consistency of oxygen-stressed reconstituted tigecycline for injection using a Hunterlab Ultrascan XE Color Spectrophotometer

Exposing samples of tigecycline to oxygen prior to reconstitution allows for development of colored degradants. An exposure of a dry tigecycline cake for five minutes, followed by reconstitution with an additional 15 minutes resting time as defined by robustness±1 minute provides for detectable degradant color formation.

Each vial of tigecycline is exposed to air for five minutes, by removing the metal cap and rubber stopper then reconstituted with 5.3 mL of 0.9% Sodium Chloride for injection, and left to rest for fifteen minutes with the rubber stopper in. The reconstituted tigecycline solution is then measured in transmittance mode using a fixed path length cell (10 mm) on a HunterLab UltraScan® XE Color Spectrophotometer. Individual measurements of six reconstituted vials per batch are obtained.

Based upon the measured CIE indices of L*, a*, b*, the calculated color value is determined using the following developed discriminator equation: 2.19×L*+2.59×a*−0.48×b*. In short, the discriminator equation and calculated color value are the same.

To evaluate the batch-to-batch consistency of oxygen-stressed reconstituted tigecycline the analyst is directed to reconstitute tigecycline samples with a defined period of oxygen exposure. The samples are then measured in transmittance mode using a fixed path length cell (10 mm) on a HunterLab UltraScan XE Color Spectrophotometer. Based upon the measured CIE indices of L*, a*, b*, a calculation using the following discriminator equation is derived that determines whether or not a tigecycline lot is of good color quality:

Calculated color value: 2.19×L*+2.59×a*−0.48×b* is <150.

The acceptance criterion was defined such that a tigecycline lot would be determined to have unacceptable color if the calculated color value was below 150.

The calculated color values and acceptability criterion is based upon a statistical analysis of ten lots of tigecycline for injection 50 mg/vial and was confirmed for three lots of tigecycline (tigecycline for injection 50 mg/vial(Lactose Formulation)).

The measured CIE indices of L*, a*, b*, with a corresponding calculation using a discriminator equation that classifies if a tigecycline lot meets color quality criteria:

Calculated Color Value: 2.19×L*+2.59×a*−0.48×b* is <150.

This derived calculated color value equation and acceptability criterion are based upon a statistical analysis of ten lots of tigecycline (tigecycline for injection 50 mg/vial). This model used 10 lots, 3 of which were lots having poor color quality while the remaining were of good color quality. The objective for the model development is to establish acceptance criteria to classify whether a lot is acceptable based on values L*, a*, and b* derived by the assay. The tigecycline lot color quality is an excellent predictor of how labile tigecycline lots may be in the end use application.

Classifying an acceptable batch as unacceptable or classifying an unacceptable batch as acceptable are two types of misclassifications. A model was derived by equating the ratio of the two types of misclassifications to the ratio of the population proportion of unacceptable lots to acceptable ones. The discriminator was developed using the minimum Expected Cost of Misclassification Rule resulting in an equation of the form: (k1*L+k2*a+k3*b). Model was developed using 3 lots defined as poor performing and 7 other lots defined as good performing with regards to color formation. This mathematical equation allows identification of good and poor performing lots. Using the above general mathematical approach (i.e. expected cost of misclassification) we generate for tigecycline the following:

Calculated Color Value: 2.19×L*+2.59×a*−0.48×b*<150.

So the developed model defines K1=2.19, K2=2.59 and K3=−0.48. Note calculated color value=discriminator with measured CIEL*a*b* values.

Instrumental color measurements are used to obtain repeatable numeric values that correspond to visual assessment. The color spectrophotometer uses the entire visible spectrum of light that is transmitted through, or reflected from a sample.

In general, color measurements can be performed in either transmission or reflection. Transmission measurement involves light passing through a liquid filled transparent cell. Reflection involves light scattering from an opaque standard with values in the middle of the visible color range. Mathematical tables representative of the human eye's color sensitivity and the color output of different light sources are used to calculate color indices.

The CIE L*a*b* color scale provides a three dimensional, linear, color scale that is organized in a cube form. The L* axis corresponds to a z axis. The maximum L* value is 100, which represents a perfect white reflecting diffuser (or clear transmission), and the minimum value is 0, which represents total light absorption (black). The a* and b* indices have no specific numerical limits.

Positive a* is red while negative a* is green. Positive b* is yellow while negative b* is blue. The method of sample preparation, sample presentation and instrument geometry must be controlled in order to maximize the precision and accuracy of the measurement.

### Spectrophotometer Standardization

The suitability of the HunterLab UltraScan® XE color spectrophotometer is performed prior to the measurement of the tigecycline sample(s) and includes both blank standardization and a green tile test to assess transmission and midrange reflection measurement effectiveness. The green tile test is a standard test, which is used to assess the stability of the color system. In general, color measurements can be performed in either transmission or reflection. Transmission measurement involves light passing through a liquid filled transparent cell. Reflection involves light scattering from an opaque standard with values in the middle of the visible color range.

### ACCURACY

The accuracy is based upon three measurements on three different color reference solutions: J, L and O (Fluka, Colour Reference Solutions acc. To USP, 2 mL, product no. 87576). The acceptance criteria are that the mean b* values obtained for three different color reference solutions J, L, O differ among themselves by more than 5 measured units. These reference standards are designed to have distinct b*, yellow-blue scale. The technique demonstrates the ability to accurately distinguish the intensity of color among the three standards, which are representative of the expected sample color range.

### Precision

### Repeatability

The repeatability is based upon twelve measurements of Fluka color reference solution “L” (Fluka, Colour Reference Solutions acc. To USP, 2 mL, product no. 87576). Color reference solution “L” is measured on two different days (N=6 measurements each day). The acceptance criteria, using the reported CIE L*A*B* values for the “L” Fluka color reference solutions, are that the % RSD (CV) be not more than 3.0%.

### Precision

### Intermediate

The intermediate precision is based upon a total of eighteen measurements of each of three different batches of tigecycline for injection 50 mg/vial. Each of the three batches (WT018, A93983, B29159) had three vials independently tested.

Three batches each measured on three different days by two different analysts. Batches included 1^{st} generation tigecycline WT018, A93983 and B29159.

The mean CIE L*a*b* indices and the values calculated. (2.19×L*+2.59×a*−0.48×b*) were compared. The mean CIE L*a*b* values for each batch are within 5 units for L*, 3 units for a* and 6 units for b*. The mean for each batch for the calculated value are within 10 units between the independent analysis. RSD (%) results for each value for each independent analysis are less than 6.0% for batches A93983 and B29159 and 12% for batch WT018.

### Specificity

The specificity is based upon the comparison of the CIE L*a*b* color indices of five color reference solutions (Fluka, Colour Reference Solutions acc. To USP, 2 mL, product no. 87576). Five individual standards (F, J, L, O, Q), which vary in color, are measured to demonstrate the variance in color CIE L*a*b* indices. The acceptance criteria is that each of the five (F, J, L, O, Q) Fluka color reference solutions have distinctive CIE L*a*b* color indices.

### Robustness

The robustness will be determined using tigecycline for Injection 50 mg/vial batch A93983. The variables will include measurement time on reconstituted tigecycline (15 min and 15 min±2 min) and saline solution volume (5.3 ml and 5.3 ml±0.1 ml).

The above described testing demonstrates the accuracy, precision, specificity, and robustness of the CIE, L*a*b* technique with regard to tigecycline for injection 50 mg/vial based on the described method.

Robustness is how well a method can tolerate minor variations in its parameters or procedures.

### Experimental 3 Color Measurement Method for Tigecycline

This color measurement method is applicable to transparent, colored solutions of tigecycline to evaluate batch-to-batch consistency of oxygen-stressed reconstituted tigecycline. Tigecycline includes tigecycline for injection 50 mg/vial as well as tigecycline for injection 50 mg/vial (lactose formulation).

Transparent, reconstituted tigecycline solution is measured in transmittance mode using a fixed path length (10 mm cuvette) on a color spectrophotometer to assess the CIE indices of L*, a*, b*. The color of the solution is measured relative to reagent-grade water using a color spectrophotometer. Suitable instruments include a HunterLab UltraScan® XE or HunterLab UltraScan® Pro Color Spectrophotometer.

### Spectrophotometers

HunterLab UltraScan® XE Color Spectrophotometer. The UltraScan XE is a color spectrophotometer with a transmission holder (CMR 2375) for cuvettes available from HunterLab Inc.

HunterLab UltraScan® Pro Color Spectrophotometer. The UltraScan XE is a color spectrophotometer with a semi-micro cell holder and optical assembly (HL#L02-1012-202) for cuvettes available from HunterLab Inc.

Cells/cuvettes—Fixed transmission cells, 10 mm path length. Cells suitable for the color instrumentation are available from Hunterlab or equivalent. The cells should be standard spectrophotometric cells which includes quartz for maximum wavelength transmission.

23-gauge needle—For use with syringe capable of holding 5.3 mL saline solution with a 10 mL syringe.

### Reagents and Materials

Reference Water—Distilled water treated by a MILLI-Q water purification system, or equivalent reagent-grade water.

Saline—0.9% Sodium Chloride injection, USP.

### Equipment Preparation

HunterLab UltraScan® XE or HunterLab UltraScan® Pro Color Spectrophotometer

Setup instrument using a 10 mm path length cell.

Install transmission holder (CMR 2375 for Hunterlab Ultrascan XE or HL#L02-1012-202 for Hunterlab Ultrascan Pro) for cuvettes (10 mm×10 mm) in the transmission compartment.

Configure measurement by selecting the following software parameters

Select TTRAN which is in the software menu for total transmission.
Small area of view;
0.375-inch port size for the HunterLab UltraScan® XE or a 0.39-inch port for the HunterLab UltraScan® Pro (Use appropriate reflectance port disc); and
UV filter nominal

### Configure Measurement by Selecting the Following Software Options

The color data should include the following:

Scale=CIE L*A*B* using L*, a*, b*; and

### D65/10° (Illuminant/Observer)

### System Suitability and Standardization

System suitability and standardization must be performed to demonstrate that the instrument is performing as per instrument specifications.

Note that system suitability includes a mid-range reflectance performance test (using green calibration tile) and standardization using water wherein the green tile test is one of the standard color instrument performance tests.

### Further, Re-Standardize Using Water after Running Each Sample Batch

### Sample Preparation Using the Following Steps

1. Open vial of tigecycline by removing plastic flip-top, aluminum band and stopper and exposing contents to laboratory air for 5 minutes;
2. Reconstitute by injecting 5.3 mL saline into the vial using a syringe and 23-gauge needle; and
3. Replace stopper on vial and let solution sit for 15 minutes. Color measurement must be performed immediately after 15 minute time period.

### Procedure for Color Measurement

1. Clean and fill the 10 mm transmission cell with the sample solution.
2. Using the transmission cell holder closest to the lens measure the sample color (press Sample icon).
3. Save results in a computer database by pressing Save Job or Save Job As under File. Be sure to use a unique identifier.

### Calculations

Using the L*, a* and b* color scale indices values insert said values in the following equation and calculate the color value:

Calculated Color Value=2.19×L*+2.59×a*−0.48×b*

Report and compare the calculated color value(s) and compare to a passing color value is 150 or above.

### Experimental

Instrumental color measurements are used to obtain repeatable numeric values that correspond to visual assessment.

The color spectrophotometer uses the entire visible spectrum of light that is

transmitted through, or reflected from a sample.

Mathematical tables representative of the human eye's color sensitivity and the color output of different light sources are used to calculate color indices.

The CIE L*a*b* color scale provides a three dimensional, linear, color scale that is organized in a cube form. The L* axis runs from top to bottom. The maximum L* value is 100, which represents a perfect reflecting diffuser, and the minimum value is 0, which represents total light absorption. The a* and b* indices have no specific numerical limits. Positive a* is red while negative a* is green. Positive b* is yellow while negative b* is blue.

The method of sample preparation, sample presentation and instrument geometry must be controlled in order to maximize the precision and accuracy of the measurement. Controlled items include:
1. Sample preparation controlled by analyst.
2. Sample presentation and instrument geometry controlled by use of specific instrument with correct cell and software parameters.

A color spectrophotometer is used to measure the color of reconstituted tigecycline solutions. The spectrophotometer is used to evaluate batch-to-batch consistency of oxygen stressed reconstituted tigecycline. Exposing samples to oxygen prior to reconstitution allows for development of colored degradants. Exposure of a dry tigecycline cake for five minutes, followed by reconstitution with an additional resting time enabled detectable degradant color formation.

Transparent, reconstituted tigecycline solutions are measured in transmittance mode using a fixed path length (10 mm cuvette) on a HunterLab UltraScan® XE Color Spectrophotometer to assess the CIE indices of L*, a*, b*.

Reconstituted tigecycline solutions are transferred to a 1 cm path-length cell and the CIE L*A*B* color indices measured.

### Accuracy

The mean “b*” values obtained for three different color reference solutions J, L, and O, differ among each other by no less than 5 units as shown in Table A. This demonstrates that the method accurately distinguishes the intensity of color between the three standards, which are representative of the expected sample color range.

TABLE A
Accuracy Data of Fluka Reference Solutions

| Fluka Reference | | |
|---|---|---|
| Solution | Sample Number | b* Value |
| J | 1 | 45.14 |
| J | 2 | 45.14 |
| J | 3 | 45.08 |
| Mean | | 45.12 |
| L | 1 | 52.07 |
| L | 2 | 51.97 |
| L | 3 | 52.00 |
| Mean | | 52.01 |
| O | 1 | 60.58 |
| O | 2 | 60.36 |
| O | 3 | 60.18 |
| Mean | | 60.37 |

### Precision

### Repeatability

The repeatability is based upon twelve measurements of Fluka color reference solution “L” (Fluka, Colour Reference Solutions acc. To USP, 2 mL, product no. 87576) and is summarized in Table B. Measured the color reference solution “L” on two different days (N=6 measurements each day). The acceptance criteria, using the reported CIE L*A*B* values for the “L” Fluka color reference solutions, are that the % RSD (CV) be not more than 3.0%. The % RSD for the L*, a* and b* color values are 0.08%, −0.49% and 0.82%, respectively.

TABLE B
Precision/Repeatability Data for the Fluka Color Reference
Solution “L”

| | Sample | | | |
|---|---|---|---|---|
| Day | Number | L* | a* | b* |
| 1 | 1 | 94.23 | −8.06 | 52.07 |
| | 2 | 94.24 | −8.06 | 51.97 |
| | 3 | 94.25 | −8.07 | 52.00 |
| | 4 | 94.24 | −8.05 | 51.97 |
| | 5 | 94.24 | −8.07 | 51.96 |
| | 6 | 94.24 | −8.05 | 51.95 |
| 2 | 1 | 94.37 | −7.98 | 51.18 |
| | 2 | 94.38 | −7.97 | 51.12 |
| | 3 | 94.38 | −7.99 | 51.18 |
| | 4 | 94.39 | −7.99 | 51.17 |
| | 5 | 94.39 | −8.00 | 51.20 |
| | 6 | 94.39 | −7.99 | 51.23 |
| Mean | | 94.31 | −8.02 | 51.58 |
| SD | | 0.08 | 0.04 | 0.42 |
| RSD | | 0.08 | −0.49 | 0.82 |
| (%) | | | | |

### Precision

### Intermediate

The intermediate precision is based upon a total of eighteen measurements of each of three different batches of tigecycline for injection 50 mg/vial. Each of the three batches (WT018, A93983, B29159) had three vials independently tested on each of three days (As shown in Tables C, D, E, F and G). The mean CIE L*a*b* indices as well as the values calculated as per the equation (2.19×L*+2.59×a*−0.48×b*), were compared. The mean CIE L*a*b* values for each batch are within 5 units for L*, 3 units for a* and 6 units for b* between the two analysts. The mean for each batch for the calculated value should be within 10 units between each independent analysis. RSD (%) results for each value for each analyst are not more than 6.0% for batches A93983 and B29159 and 12% for batch WT018. The mean calculated difference between independent analysis are: 0.0773 for batch A93983, 2.62 for batch B29159, and 2.8362 for batch WT018.

TABLE C
Intermediate Precision Data for Batch A93983

| | | | | | | Calculated |
|---|---|---|---|---|---|---|
| | Independent | | | | | Color |
| Day | Analysis^{a} | Vial # | L* | a* | b* | Value^{b} |
| 1 | 1 | 1 | 81.54 | 18.76 | 131.47 | 164.05 |
| | | 2 | 81.57 | 18.93 | 131.34 | 163.99 |
| | | 3 | 81.41 | 19.17 | 131.44 | 164.85 |
| 2 | 1 | 1 | 81.63 | 18.90 | 129.89 | 165.37 |
| | | 2 | 81.54 | 18.75 | 130.33 | 164.57 |
| | | 3 | 81.69 | 18.59 | 131.18 | 164.08 |
| 3 | 1 | 1 | 81.65 | 18.77 | 130.84 | 164.62 |
| | | 2 | 81.84 | 18.65 | 129.01 | 165.61 |
| | | 3 | 81.66 | 18.74 | 131.57 | 164.23 |
| Mean | | | 81.61 | 18.81 | 130.79 | 164.67 |
| SD | | | 0.12 | 0.18 | 0.88 | 0.54 |
| RSD | | | 0.16 | 0.95 | 0.68 | 0.33 |
| 1 | 2 | 1 | 81.84 | 18.65 | 131.15 | 164.58 |
| | | 2 | 81.88 | 18.78 | 131.36 | 164.91 |
| | | 3 | 81.32 | 19.38 | 130.55 | 165.62 |
| 2 | 2 | 1 | 82.12 | 18.51 | 131.42 | 164.70 |
| | | 2 | 81.57 | 18.95 | 131.04 | 164.82 |
| | | 3 | 81.49 | 18.17 | 129.9 | 163.17 |
| 3 | 2 | 1 | 81.71 | 18.68 | 129.69 | 165.07 |
| | | 2 | 81.90 | 18.37 | 128.56 | 165.23 |
| | | 3 | 81.56 | 18.77 | 130.78 | 164.46 |
| Mean | | | 81.71 | 18.70 | 130.46 | 164.75 |
| SD | | | 0.25 | 0.35 | 0.95 | 0.69 |
| RSD | | | 0.30 | 1.87 | 0.73 | 0.42 |
| Difference^{c} | | | 0.10 | 0.11 | 0.33 | 0.08 |
| Limit^{d} | | | 5 | 3 | 6 units | 10 units |
| | | | units | units | | |

^{a}Hunterlab UltraScan XE color spectrophotometer
^{b}Equation used to determine the calculated color value: 2.19 × L* + 2.59 × a* − 0.48 × b*
^{c}Difference = (Mean color value of Analyst 1 − Mean color value of Analyst 2)
^{d}Limit is defined as the acceptable number of units in difference between the two analysts for each measured and calculated color value.

TABLE D
Intermediate Precision Data for Batch B29159

| | | | | | | Calculated |
|---|---|---|---|---|---|---|
| | Independent | | | | | Color |
| Day | Analysis^{a} | Vial # | L* | a* | b* | Value^{b} |
| 1 | 1 | 1 | 79.42 | 18.57 | 128.37 | 160.41 |
| | | 2 | 79.04 | 18.64 | 127.60 | 160.13 |
| | | 3 | 77.37 | 17.64 | 125.42 | 154.93 |
| 2 | 1 | 1 | 78.13 | 18.01 | 125.48 | 157.52 |
| | | 2 | 73.78 | 15.91 | 117.24 | 146.51 |
| | | 3 | 77.69 | 18.28 | 125.38 | 157.30 |
| 3 | 1 | 1 | 77.95 | 18.24 | 125.38 | 157.77 |
| | | 2 | 77.10 | 17.98 | 124.64 | 155.59 |
| | | 3 | 78.05 | 18.05 | 123.76 | 158.27 |
| Mean | | | 77.61 | 18.15 | 125.81 | 156.59 |
| SD | | | 1.64 | 1.08 | 4.15 | 4.45 |
| RSD | | | 2.11 | 5.96 | 3.30 | 2.84 |
| 1 | 2 | 1 | 78.25 | 17.69 | 123.31 | 157.99 |
| | | 2 | 79.54 | 18.71 | 126.66 | 161.55 |
| | | 3 | 79.81 | 18.52 | 125.6 | 162.46 |
| 2 | 2 | 1 | 77.26 | 18.06 | 125.06 | 155.95 |
| | | 2 | 78.26 | 18.42 | 125.82 | 158.70 |
| | | 3 | 79.05 | 18.77 | 127.46 | 160.55 |
| 3 | 2 | 1 | 79.87 | 18.75 | 128.78 | 159.74 |
| | | 2 | 77.98 | 18.24 | 125.38 | 157.84 |
| | | 3 | 77.50 | 17.85 | 125.32 | 155.80 |
| Mean | | | 78.61 | 18.33 | 125.93 | 159.21 |
| SD | | | 0.99 | 0.40 | 1.56 | 2.53 |
| RSD | | | 1.26 | 2.18 | 1.24 | 1.59 |
| Difference^{c} | | | 1.00 | 0.18 | 0.12 | 2.62 |
| Limit^{d} | | | 5 | 3 | 6 units | 10 units |
| | | | units | units | | |

^{a}Instrument: Hunterlab UltraScan XE color spectrophotometer
^{b}Equation used to determine the calculated color value: 2.19 × L* + 2.59 × a* − 0.48 × b*
^{c}Difference = (Mean color value of Analyst 1 − Mean color value of Analyst 2)
^{d}Limit is defined as the acceptable number of units in difference between the two analysts for each measured and calculated color value.

TABLE E
Intermediate Precision Data for Batch WT018

| | | | | | | Calculated |
|---|---|---|---|---|---|---|
| | Independent | | | | | Color |
| Day | analysis^{a} | Vial # | L* | a* | b* | Value^{b} |
| 1 | 1 | 1 | 74.16 | 16.28 | 117.56 | 148.15 |
| | | 2 | 73.70 | 16.12 | 119.40 | 145.79 |
| | | 3 | 75.38 | 16.91 | 122.12 | 150.26 |
| 2 | 1 | 1 | 73.43 | 16.49 | 119.40 | 146.21 |
| | | 2 | 75.21 | 16.66 | 121.23 | 149.67 |
| | | 3 | 75.30 | 17.04 | 121.58 | 150.68 |
| 3 | 1 | 1 | 72.32 | 15.69 | 117.45 | 142.64 |
| | | 2 | 72.12 | 15.72 | 117.35 | 142.33 |
| | | 3 | 74.77 | 16.97 | 119.59 | 150.29 |
| Mean | | | 74.04 | 16.43 | 119.52 | 147.34 |
| SD | | | 1.27 | 0.51 | 1.88 | 3.29 |
| RSD | | | 1.72 | 3.14 | 1.58 | 2.23 |
| 1 | 2 | 1 | 74.07 | 16.41 | 118.37 | 147.89 |
| | | 2 | 72.95 | 15.23 | 117.29 | 142.91 |
| | | 3 | 73.18 | 15.69 | 117.24 | 144.62 |
| 2 | 2 | 1 | 71.63 | 15.48 | 115.76 | 141.40 |
| | | 2 | 72.17 | 15.30 | 116.32 | 141.85 |
| | | 3 | 73.14 | 16.12 | 117.71 | 145.33 |
| 3 | 2 | 1 | 72.56 | 15.71 | 114.13 | 144.82 |
| | | 2 | 74.59 | 16.54 | 120.08 | 148.55 |
| | | 3 | 72.28 | 15.88 | 117.37 | 143.08 |
| Mean | | | 72.95 | 15.82 | 117.14 | 144.51 |
| SD | | | 0.94 | 0.46 | 1.67 | 2.50 |
| RSD | | | 1.29 | 2.93 | 1.42 | 1.73 |
| Difference^{c} | | | 1.09 | 0.61 | 2.38 | 2.83 |
| Limit^{d} | | | 5 | 3 | 6 units | 10 units |
| | | | units | units | | |

^{a}Two independent analysts Instrument: Hunterlab UltraScan XE color spectrophotometer
^{b}Equation used to determine the calculated color value: 2.19 × L* + 2.59 × a* − 0.48 × b*
^{c}Difference = (Mean color value of Analyst 1 − Mean color value of Analyst 2)
^{d}Limit is defined as the acceptable number of units in difference between the two analysts for each measured and calculated color value.

TABLE F
Intermediate Precision Data for Batch TR004

| | | | | | | Calculated |
|---|---|---|---|---|---|---|
| | | | | | | Color |
| Day | Analyst^{a} | Vial # | L* | a* | b* | Value^{b} |
| 1 | 1 | 1 | 84.37 | 15.97 | 130.56 | 163.46 |
| | | 2 | 84.04 | 16.08 | 130.11 | 163.24 |
| | | 3 | 84.32 | 16.07 | 130.65 | 163.57 |
| | | 4 | 84.69 | 15.98 | 130.65 | 164.15 |
| | | 5 | 84.27 | 15.74 | 129.88 | 162.98 |
| | | 6 | 84.43 | 15.97 | 130.57 | 163.59 |
| 2 | 1 | 1 | 85.01 | 16.01 | 131.75 | 164.40 |
| | | 2 | 85.15 | 15.76 | 131.86 | 164.00 |
| | | 3 | 84.98 | 15.91 | 131.26 | 164.31 |
| | | 4 | 85.20 | 15.69 | 131.56 | 164.08 |
| | | 5 | 85.05 | 15.90 | 131.68 | 164.23 |
| | | 6 | 85.36 | 15.31 | 131.46 | 163.49 |
| Mean | | | 84.74 | 15.87 | 131.00 | 163.79 |
| SD | | | 0.44 | 0.22 | 0.68 | 0.46 |
| RSD | | | 0.52 | 1.36 | 0.52 | 0.28 |
| 1 | 2 | 1 | 85.51 | 15.19 | 130.36 | 164.04 |
| | | 2 | 85.24 | 14.80 | 128.89 | 163.14 |
| | | 3 | 85.45 | 15.08 | 130.91 | 163.36 |
| | | 4 | 85.47 | 15.14 | 131.70 | 163.18 |
| | | 5 | 85.32 | 14.90 | 131.07 | 162.53 |
| | | 6 | 85.38 | 15.17 | 131.24 | 163.28 |
| 2 | 2 | 1 | 85.21 | 15.00 | 129.85 | 163.13 |
| | | 2 | 85.25 | 14.53 | 130.77 | 161.56 |
| | | 3 | 85.34 | 15.12 | 132.23 | 162.59 |
| | | 4 | 84.89 | 15.25 | 128.59 | 163.68 |
| | | 5 | 85.09 | 15.37 | 131.50 | 163.04 |
| | | 6 | 85.08 | 15.14 | 132.07 | 162.14 |
| Mean | | | 85.27 | 15.06 | 130.77 | 162.97 |
| SD | | | 0.22 | 0.22 | 1.16 | 0.70 |
| RSD | | | 0.25 | 1.49 | 0.89 | 0.43 |

| | | | | |
|---|---|---|---|---|
| Difference^{c} | 0.53 | 0.81 | 0.23 | 0.82 |
| Limit^{d} | 5 units | 3 units | 6 units | 10 units |

^{a}Analyst 1: Hunterlab UltraScan Pro color spectrophotometer, Analyst 2: Hunterlab UltraScan XE color spectrophotometer
^{b}Calculated Color Value = 2.19 × L* + 2.59 × a* − 0.48 × b*
^{c}Difference = (Mean color value of Analyst 1 − Mean color value of Analyst 2)
^{d}Limit is defined as the acceptable number of units in difference between the two analysts for each measured and calculated color value.

TABLE G
Intermediate Precision Data for Batch CR-0454/04-01 (045404A)

| | | | | | | Calculated |
|---|---|---|---|---|---|---|
| Day | Analyst^{a} | Vial # | L* | a* | b* | Color Value^{b} |
| 1 | 1 | 1 | 85.28 | 12.80 | 129.44 | 157.78 |
| | | 2 | 85.44 | 12.84 | 129.60 | 158.16 |
| | | 3 | 85.89 | 12.38 | 130.04 | 157.74 |
| | | 4 | 86.26 | 12.46 | 130.39 | 158.59 |
| | | 5 | 85.04 | 12.49 | 128.83 | 156.75 |
| | | 6 | 86.19 | 12.73 | 130.45 | 159.11 |
| 2 | 1 | 1 | 86.28 | 12.44 | 130.36 | 158.60 |
| | | 2 | 86.12 | 12.43 | 130.27 | 158.27 |
| | | 3 | 86.52 | 12.33 | 130.13 | 158.95 |
| | | 4 | 86.26 | 12.94 | 130.59 | 159.74 |
| | | 5 | 86.28 | 12.42 | 130.29 | 158.58 |
| | | 6 | 86.40 | 12.55 | 130.21 | 159.22 |
| Mean | | | 86.00 | 12.57 | 130.05 | 158.46 |
| SD | | | 0.48 | 0.20 | 0.51 | 0.88 |
| RSD | | | 0.56 | 1.63 | 0.39 | 0.50 |
| 1 | 2 | 1 | 86.02 | 12.13 | 129.62 | 157.58 |
| | | 2 | 86.31 | 12.33 | 129.78 | 158.66 |
| | | 3 | 86.23 | 12.36 | 130.45 | 158.24 |
| | | 4 | 86.43 | 12.04 | 128.65 | 158.71 |
| | | 5 | 86.31 | 12.24 | 128.83 | 158.88 |
| | | 6 | 86.67 | 12.03 | 129.59 | 158.76 |
| 2 | 2 | 1 | 86.15 | 11.89 | 129.79 | 157.16 |
| | | 2 | 86.23 | 11.97 | 129.16 | 157.85 |
| | | 3 | 85.91 | 12.70 | 130.32 | 158.48 |
| | | 4 | 86.39 | 11.93 | 129.20 | 158.08 |
| | | 5 | 86.47 | 11.95 | 130.75 | 157.56 |
| | | 6 | 86.27 | 11.98 | 130.56 | 157.29 |
| Mean | | | 86.28 | 12.13 | 129.73 | 158.11 |
| SD | | | 0.20 | 0.24 | 0.70 | 0.69 |
| RSD | | | 0.23 | 1.97 | 0.54 | 0.44 |
| Difference^{c} | | | 0.28 | 0.44 | 0.32 | 0.35 |
| Limit^{d} | | | 5 | 3 | 6 units | 10 units |
| | | | units | units | | |

^{a}Analyst 1: Hunterlab UltraScan Pro color spectrophotometer, Analyst 2: Hunterlab UltraScan XE color spectrophotometer.
^{b}Calculated Color Value = 2.19 × L* + 2.59 × a* − 0.48 × b*
^{c}Difference = (Mean color value of Analyst 1 − Mean color value of Analyst 2)
^{d}Limit is defined as the acceptable number of units in difference between the two analysts for each measured and calculated color value.

### Specificity

The specificity is based upon the comparison of the CIE L*a*b* color indices of five color reference solutions (Fluka, Colour Reference Solutions acc. To USP, 2 mL, product no. 87576). Five individual standards (F, J, L, O, Q), which vary in color, were measured to demonstrate the variance in color CIE L*a*b* indices (As shown in Table H). The acceptance criteria is each of the five (F, J, L, O, Q) Fluka color reference solutions show distinctive CIE L*a*b*color indices.

TABLE H
SPECIFICITY DATA USING FLUKA REFERENCE SOLUTIONS
REFERENCE STANDARD SAMPLE # L* A* B*
Specificity Data using Fluka Reference Solutions

| Reference | | | | |
|---|---|---|---|---|
| Standard | Sample # | L* | a* | b* |
| L | 1 | 94.23 | −8.06 | 52.07 |
| | 2 | 94.24 | −8.06 | 51.97 |
| | 3 | 94.25 | −8.07 | 52.00 |
| Mean | | 94.24 | −8.06 | 52.01 |
| F | 1 | 98.14 | −3.75 | 15.78 |
| | 2 | 98.14 | −3.74 | 15.73 |
| | 3 | 98.14 | −3.74 | 15.73 |
| Mean | | 98.14 | −3.74 | 15.75 |
| J | 1 | 96.09 | −11.29 | 45.14 |
| | 2 | 96.10 | −11.29 | 45.14 |
| | 3 | 96.10 | −11.28 | 45.08 |
| Mean | | 96.10 | −11.29 | 45.12 |
| O | 1 | 96.96 | −16.55 | 60.58 |
| | 2 | 96.97 | −16.54 | 60.36 |
| | 3 | 96.99 | −16.53 | 60.18 |
| Mean | | 96.97 | −16.54 | 60.37 |
| Q | 1 | 99.01 | 0.50 | 2.65 |
| | 2 | 99.01 | 0.50 | 2.67 |
| | 3 | 99.01 | 0.49 | 2.68 |
| Mean | | 99.01 | 0.50 | 2.67 |

### Robustness

The robustness was determined using tigecycline for Injection 50 mg/vial batch A93983. The variables will include measurement time on reconstituted tigecycline (15 min±2 min) and saline solution volume (5.3 ml and 5.3 ml+0.1 ml) (as shown in Table I).

Robustness Data: Variation in Measurement Time and Volume of Diluent (Batch A93983) Variable L* a* b* Calculated Color Value

TABLE I
Robustness Data: Variation in Measurement Time and Volume
of Diluent (Batch A93983)

| | | | | Calculated |
|---|---|---|---|---|
| Variable | L* | a* | b* | Color Value |
| 13 minutes | 81.70 | 18.68 | 130.06 | 164.87 |
| 15 minutes | 81.23 | 19.10 | 129.70 | 165.10 |
| 17 minutes | 81.60 | 18.66 | 129.73 | 164.76 |
| 5.2 mL | 81.06 | 19.41 | 130.23 | 165.28 |
| 5.3 mL | 81.43 | 18.72 | 130.36 | 164.24 |
| 5.4 mL | 81.93 | 17.94 | 129.54 | 163.71 |

Determination of the color value of reconstituted tigecycline for Injection 50 mg/vial using a Hunterlab Ultrascan XE Color Spectrophotometer.

TABLE J
Intermediate Precision for Tigecycline (Tigecycline for injection 50 mg/Vial):
Comparisons of the Mean, Standard Deviations, and CV's

| | | | Analyst 1 | | | Analyst 2 | | |
|---|---|---|---|---|---|---|---|---|
| Batch | Item^{a} | Source | Mean | Std dev | CV (%) | Mean | Std dev | CV (%) |
| A93983 | L | Day | | 0.0000 | 0.00 | | 0.0913 | 0.11 |
| | | Sample | | 0.2481 | 0.30 | | 0.0901 | 0.11 |
| | | Total | 81.7100 | 0.2481 | 0.30 | 81.6144 | 0.1283 | 0.16 |
| | a | Day | | 0.0766 | 0.41 | | 0.0922 | 0.49 |
| | | Sample | | 0.3409 | 1.82 | | 0.1532 | 0.81 |
| | | Total | 18.6956 | 0.3494 | 1.87 | 18.8067 | 0.1787 | 0.95 |
| | b | Day | | 0.5301 | 0.41 | | 0.2391 | 0.18 |
| | | Sample | | 0.7905 | 0.61 | | 0.8513 | 0.65 |
| | | Total | 130.4611 | 0.9518 | 0.73 | 130.7856 | 0.8842 | 0.68 |
| | Value | Day | | 0.1176 | 0.07 | | 0.0000 | 0.00 |
| | | Sample | | 0.6761 | 0.41 | | 0.5415 | 0.33 |
| | | Total | 164.7451 | 0.6862 | 0.42 | 164.6678 | 0.5415 | 0.33 |
| B29159 | L | Day | | 0.0000 | 0.00 | | 0.5330 | 0.69 |
| | | Sample | | 0.9868 | 1.26 | | 1.5487 | 2.00 |
| | | Total | 78.6133 | 0.9868 | 1.26 | 77.6144 | 1.6379 | 2.11 |
| | a | Day | | 0.0000 | 0.00 | | 0.3560 | 1.96 |
| | | Sample | | 0.3999 | 2.18 | | 1.0208 | 5.63 |
| | | Total | 18.3344 | 0.3999 | 2.18 | 18.1467 | 1.0812 | 5.96 |
| | b | Day | | 0.0000 | 0.00 | | 1.5208 | 1.21 |
| | | Sample | | 1.5578 | 1.24 | | 3.8670 | 3.07 |
| | | Total | 125.9322 | 1.5578 | 1.24 | 125.8078 | 4.1553 | 3.30 |
| | Value | Day | | 0.0000 | 0.00 | | 0.0000 | 0.00 |
| | | Sample | | 2.5318 | 1.59 | | 4.4533 | 2.84 |
| | | Total | 159.2090 | 2.5318 | 1.59 | 156.5878 | 4.4533 | 2.84 |
| WT018 | L | Day | | 0.2052 | 0.28 | | 0.5243 | 0.71 |
| | | Sample | | 0.9173 | 1.26 | | 1.1610 | 1.57 |
| | | Total | 72.9522 | 0.9400 | 1.29 | 74.0433 | 1.2738 | 1.72 |
| | a | Day | | 0.0000 | 0.00 | | 0.0595 | 0.36 |
| | | Sample | | 0.4641 | 2.93 | | 0.5123 | 3.12 |
| | | Total | 15.8178 | 0.4641 | 2.93 | 16.4311 | 0.5157 | 3.14 |
| | b | Day | | 0.0000 | 0.00 | | 0.8979 | 0.75 |
| | | Sample | | 1.6660 | 1.42 | | 1.6568 | 1.39 |
| | | Total | 117.1411 | 1.6660 | 1.42 | 119.5200 | 1.8845 | 1.58 |
| | Value | Day | | 0.0000 | 0.00 | | 0.7342 | 0.50 |
| | | Sample | | 2.5036 | 1.73 | | 3.2012 | 2.17 |
| | | Total | 144.5057 | 2.5036 | 1.73 | 147.3419 | 3.2843 | 2.23 |

^{a}Value = 2.19 × L* + 2.59 × a* − 0.48 × b*

TABLE K
Intermediate Precision for Tigecycline (Tigecycline for injection
50 mg/Vial, Lactose Formulation): Comparisons of the Mean, Standard
Deviations, and CV's from Statistical Report SRN07-049

| | | | Analyst 1/WP | | | Analyst 2/AQS | | |
|---|---|---|---|---|---|---|---|---|
| Batch | Item^{a} | Source | Mean | Std dev | CV (%) | Mean | Std dev | CV (%) |
| TR004 | L* | Day | | 0.5406 | 0.64 | | 0.1695 | 0.20 |
| | | Sample | | 0.1809 | 0.21 | | 0.1331 | 0.16 |
| | | Total | 84.7392 | 0.5701 | 0.67 | 85.2692 | 0.2155 | 0.25 |
| | a* | Day | | 0.1207 | 0.76 | | 0.0000 | 0.00 |
| | | Sample | | 0.1967 | 1.24 | | 0.2247 | 1.49 |
| | | Total | 15.8658 | 0.2308 | 1.45 | 15.0575 | 0.2247 | 1.49 |
| | b* | Day | | 0.8350 | 0.64 | | 0.0000 | 0.00 |
| | | Sample | | 0.2770 | 0.21 | | 1.1619 | 0.89 |
| | | Total | 130.9992 | 0.8797 | 0.67 | 130.7650 | 1.1619 | 0.89 |
| | Value | Day | | 0.3880 | 0.24 | | 0.3008 | 0.18 |
| | | Sample | | 0.3613 | 0.22 | | 0.6367 | 0.39 |
| | | Total | 163.7917 | 0.5301 | 0.32 | 162.9712 | 0.7042 | 0.43 |
| CR- | L* | Day | | 0.4168 | 0.48 | | 0.0000 | 0.00 |
| 04540401 | | | | | | | | |
| | | Sample | | 0.3688 | 0.43 | | 0.2025 | 0.23 |
| | | Total | 85.9967 | 0.5565 | 0.65 | 86.2825 | 0.2025 | 0.23 |
| | a* | Day | | 0.0000 | 0.00 | | 0.0000 | 0.00 |
| | | Sample | | 0.2045 | 1.63 | | 0.2386 | 1.97 |
| | | Total | 12.5675 | 0.2045 | 1.63 | 12.1292 | 0.2386 | 1.97 |
| | b* | Day | | 0.3148 | 0.24 | | 0.1950 | 0.15 |
| | | Sample | | 0.4543 | 0.35 | | 0.6733 | 0.52 |
| | | Total | 130.0500 | 0.5527 | 0.42 | 129.7250 | 0.7010 | 0.54 |
| | Value | Day | | 0.5477 | 0.35 | | 0.4799 | 0.30 |
| | | Sample | | 0.6849 | 0.43 | | 0.4931 | 0.31 |
| | | Total | 158.4585 | 0.8770 | 0.55 | 158.1052 | 0.6881 | 0.44 |

^{a}Value = 2.19 × L* + 2.59 × a* − 0.48 × b*

### Experimental 4

Demonstration of changes in measured color and the calculated color value as they relate to the impurity profile of tigecycline after spiking with a known blue colored degradant, Compound I

The spiking concentrations required to produce a clear color change in tigecycline (tigecycline for injection 50 mg/vial (lactose formulation)) was characterized using both a Hunterlab UltraScan XE color spectrophotometer and a quantitative HPLC method. The threshold for visual green color resulting from spiking with Compound I colored degradant was determined for each category of tigecycline samples. An excellent linear correlation (R>0.99) between the concentration of Compound I and the calculated color value is observed. The spiking of tigecycline with ca. 0.045 concentration Compound I results in observation of a slightly green color in the reconstituted tigecycline and tigecycline API. This green color is more pronounced at the higher Compound I concentration. Tables 1, 2, 3 and 4 summarize the calculated color values and HPLC results for tigecycline Registration stability batches (CR0040-50-01, CR0040-50-02, CR0040-50-03), validation stability batches (B41133, B41404, B41710) and API used in the validation stability batches (3000002665, 2000140691, 2000140693). As per the color methods employed the calculated color value is calculated from the measured CIEL*a*b* values using the equation below:

Calculated Color Value=2.19×L*+2.59×a*−0.48×b*

TABLE 1
Summary of Calculated Color Value and the HPLC results for Registration Stability
batches Stored at 25° C.

| | | No Spike | | Spike Level 1 | | Spike Level 2 | |
|---|---|---|---|---|---|---|---|
| | | Measured | Measured | Measured | Measured | Measured | Measured |
| Batch | Vial | RRT 0.36 | Color Value | RRT 0.36 | Color Value | RRT 0.36 | Color Value |
| CR0040-50-01 | 1 | 0.000 | 160.3 | 0.049 | 146.7 | 0.095 | 136.3 |
| | 2 | 0.000 | 161.6 | 0.043 | 148.2 | 0.102 | 135.6 |
| | 3 | 0.000 | 161.2 | 0.045 | 146.7 | 0.084 | 134.9 |
| CR0040-05-02 | 1 | 0.000 | 163.5 | 0.043 | 149.1 | 0.090 | 134.9 |
| | 2 | 0.000 | 163.0 | 0.042 | 149.3 | 0.077 | 137.0 |
| | 3 | 0.000 | 162.0 | 0.044 | 153.2 | 0.076 | 141.1 |
| CR0040-05-03 | 1 | 0.000 | 160.6 | 0.044 | 149.7 | 0.094 | 134.7 |
| | 2 | 0.000 | 160.5 | 0.040 | 149.2 | 0.092 | 135.7 |
| | 3 | 0.000 | 160.5 | 0.035 | 148.4 | 0.074 | 136.1 |

TABLE 2
Summary of Calculated Color Value and the HPLC results for Registration
Stability batches Stored at 5° C.

| | No Spike | | Spike Level 1 | | Spike Level 2 | |
|---|---|---|---|---|---|---|
| | Measured | | | Measured | Measured | |
| | RRT | Measured | Measured | Color | RRT | Measured |
| Batch | 0.36 | Color Value | RRT 0.36 | Value | 0.36 | Color Value |
| CR0040-50-01 | 0 | 159.8 | 0.047 | 147.7 | 0.097 | 134.6 |
| CR0040-05-02 | 0 | 162.2 | 0.047 | 149.7 | 0.094 | 137.9 |
| CR0040-05-03 | 0 | 160.5 | 0.044 | 147.9 | 0.102 | 134.7 |

TABLE 3
Summary of Calculated Color Value and the HPLC results for Validation
Stability batches Stored at 25° C.

| | | | | Spike Level 1 | | | |
|---|---|---|---|---|---|---|---|
| | | No Spike | | | Measured | Spike Level 2 | |
| | | Measured | Measured | Measured | Color | Measured | Measured |
| Batch | Vial | RRT 0.36 | Color Value | RRT 0.36 | Value | RRT 0.36 | Color Value |
| B41133 | 1 | 0 | 157.6 | 0.040 | 147.4 | 0.083 | 132.0 |
| | 2 | — | — | 0.048 | 147.7 | 0.087 | 132.2 |
| B41404 | 1 | 0 | 156.2 | 0.042 | 145.2 | 0.091 | 133.7 |
| | 2 | — | — | 0.042 | 145.3 | 0.091 | 132.9 |
| B41710 | 1 | 0 | 155.2 | 0.042 | 144.2 | 0.098 | 131.6 |
| | 2 | — | — | 0.040 | 144.8 | 0.095 | 132.2 |

TABLE 4
SUMMARY OF CALCULATED COLOR VALUE AND THE HPLC RESULTS
FOR API USED IN VALIDATION STABILITY BATCHES STORED AT 5° C.

| | No Spike | | | | | |
|---|---|---|---|---|---|---|
| | | Measured | Spike Level 1 | | spike Level 2 | |
| | Measured | Color | Measured | Measured | Measured | Measured |
| Batch | RRT 0.36 | Value | RRT 0.36 | Color Value | RRT 0.36 | Color Value |
| 3000002665 | 0 | 168.4 | 0.034446 | 148.3 | 0.075464 | 129.4 |
| 2000140691 | 0 | 166.7 | 0.035744 | 145.0 | 0.082363 | 128.1 |
| 2000140693 | 0 | 167.8 | 0.035537 | 148.5 | 0.071324 | 129.9 |

### General Sample Preparation Procedure

The samples were tested immediately after reconstitution according to the label directions (5.3 ml of 0.9% saline). In addition, reconstituted samples were spiked with the blue Compound I impurity at 2 levels: the lower level was chosen at which the greenish coloration first appears visually, and the higher level was chosen where the sample solution was strong visible green. The solutions were then analyzed by the approved product purity method, to quantitate the exact level of Compound I impurity in the solution. Also, the general color method for tigecycline and tigecycline API was used to measure the color value of these solutions containing the different levels of Compound I.

### Experimental 5

### Isolation of Compound I

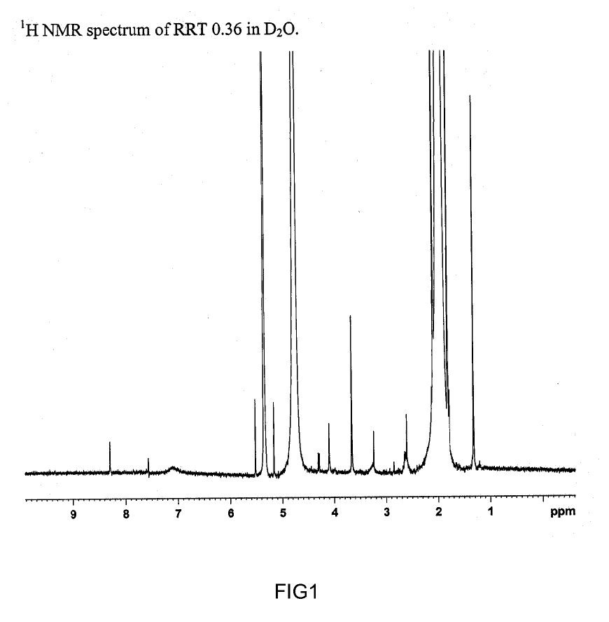

Tigecycline was degraded at room temperature by dissolving 400 mg of tigecycline in 2 mL of water or deuterated water. The solution, open to air, was stirred for a few hours until the solution changed color from yellow/orange to dark green. The degradation mixture was then subjected to a preparative HPLC separation using a Dynamax Solvent Delivery System (Model SD-1) equipped with UV detector (Model UV-1) with its wavelength set at 248 nm, and a Prodigy C₁₈(3) column (20×250 mm). The column was eluted with a step gradient of water with 50 mM ammonium acetate (mobile phase A, or deuterated water/sodium acetate-d₃ for the NMR sample) and acetonitrile (mobile phase B). The flow rate was set at 25 mL/min, beginning with 7% B for 2 min, followed by a linear gradient from 7% to 30% B over 40 min. The apex portion of the peak of interest (blue fraction, 17 min) was collected. The acetonitrile in the collected fraction was removed by extraction with methylene chloride-d₂ and the remaining aqueous layer was quickly concentrated by speedvac for 10-20 min and then immediately frozen or lyophilized to dryness for NMR or MS studies. FIG. 1 shows the NMR of Compound I.

### LC-MS and LC-MS/MS Conditions

A Shimadzu-10Advp HPLC system coupled with Applied Biosystems-PE SCIEX QSTAR PULSAR i quadrupole time-of-flight tandem mass spectrometer equipped with an electrospray ionization ion source operated in positive ion mode was used for LC-MS and LC-MS/MS experiments. The column used was Phenomenex Luna C₁₈(2) (150×4.6 mm, 3 μm). The temperature of the auto sampler was set at 8° C. and the column at 30° C. Mobile phases A and B each contained 3.85 g of ammonium acetate dissolved in 950 mL and 500 mL of water and the pH was adjusted to 6.2 with acetic acid, followed by adding 50 mL to A and 500 mL to B of acetonitrile, respectively. The linear gradient started at 85% A/15% B and ended at 57% A/43% B in 40 min. The HPLC flow rate was set at 1.0 mL/min, and the HPLC eluent was split to allow approximately 50 μL/min into the electrospray ion source of the mass spectrometer. The on-line PDA detector was used to collect the spectra from 200 to 800 nm.

### NMR Spectroscopy of Compound I

NMR samples were prepared from the lyophilized solids or from the frozen fractions in deuterated water collected from preparative HPLC. ¹H, TOCSY, HSQC and HMBC experiments were conducted on a Bruker DRX-500 spectrometer equipped with a 5 mm TXI cryo probe at 15° C. Additionally, ¹H, COSY, TOCSY, HSQC and HMBC experiments were conducted on a Bruker DRX-400 spectrometer with a 5 mm BBI z-gradient probe and on a DRX-500 spectrometer with a 4 mm LC-NMR z-gradient probe at 5° C. to 10° C.

### Results and Discussion

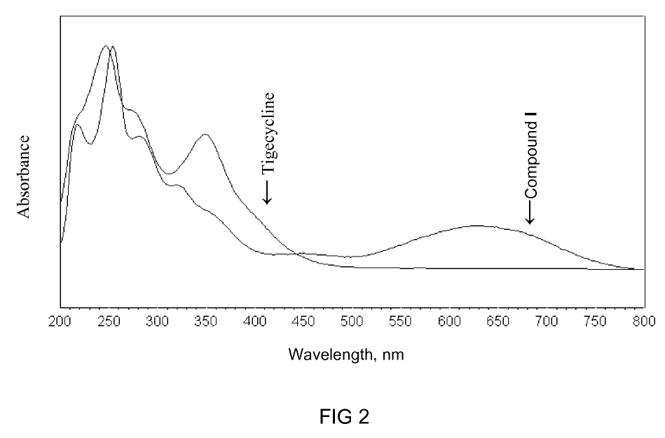

To isolate a sufficient amount of compound I, tigecycline was prepared using forced degradation technique. The intense blue colored compound I was isolated from the forced degradation sample of tigecycline by a preparative reversed phase HPLC. The compound I was then analyzed by LC-MS/MS. FIG. 2 shows the UV-VIS spectra of tigecycline that has an absorbance band at about 350 nm region only and the compound I that has an absorbance band in a visible region at 630 nm, suitable for a transmitted blue color.

The structure of compound I was determined to be an iminoquinone derivative based on detailed spectroscopic data analyses. The mass spectrum of compound I showed a positive molecular ion at m/z 616. Unlike other degradants, compound I did not show the negative molecular ion of m/z 614. The measured accurate mass of 616.2624 atomic mass units (amu) indicated that it has the ionic formula of C₂₉H₃₈N₅O₁₀ (calculated: 616.2613 amu). At the same retention time, there was another ion observed with a measured accurate mass of 572.2668 amu, corresponding to a elemental composition of C₂₈H₃₈N₅O₈ (calculated: 572.2714 amu), which could be either the fragment ion from the m/z 616 compound or a different component of the co-eluting peak. LC-MS/MS experiments were conducted with the isolated compound I on both ions at m/z 616 and m/z 572 to obtain structural information. The LC-MS/MS spectra of these two ions had the same fragmentation pattern, indicating that the ion at m/z 572 was a fragment ion of m/z 616. The difference between m/z 616 and m/z 572 corresponded to a loss of a CO₂ group, indicating that compound I contained a carboxylic acid moiety.

These fragment ions were also present in the LC-MS/MS spectrum of m/z 572, which confirmed that compound I lost the CO₂ group first, followed by further fragmentation to other ions. The presence of two strong fragment ions of m/z 232 and 247 and their elemental compositions indicating that the B-ring of tigecycline was opened to form the carboxylic acid. Two other fragment ions observed at m/z 360 and 304 also support these assignments. Other fragment ions with corresponding assignments were consistent with the structure. The LC-MS/MS fragmentation data provided the necessary structural information. By choosing two known fragment ions of m/z 86.0964 and m/z 555.2449 as references, other fragment ions with the closest fitting elemental compositions were calculated.

Compound I was studied by NMR spectroscopy as well. The freshly isolated blue fraction from preparative HPLC was lyophilized to dryness. The dried dark blue solid was dissolved in deuterated solvents including dimethylsulfoxide (DMSO), acetonitrile, and acetone for the NMR data acquisition; however, none of the NMR spectra obtained showed practically legible signals. Freshly collected preparative HPLC fraction was quickly extracted with methylene chloride to remove acetonitrile followed by speed-vacuum, and the sample in deuterated water was immediately transferred to an NMR tube to acquire spectra at 15° C. Since this compound was very unstable, there was limited time window of data collection for structural elucidation. Several prepreparative HPLC separations were performed to isolate the fresh compound I in order to obtain the key structural information through 1D and 2D NMR studies including ¹H NMR, COSY, TOCSY, HSQC and HMBC experiments. In the ¹H NMR spectrum, a singlet aromatic proton resonance was detected at δ_{H} 8.28, indicating that H-8 was present. A highly intense singlet peak at δ_{H} 3.59 was assigned to methyl protons of the C-7 dimethyl amino group, and their associated carbons were detected at δ_{C} 47.1 in the HSQC spectrum. The proton chemical shifts of these two methyl groups were significantly shifted downfield when compared with that of tigecycline. In the HMBC spectrum, these methyl protons showed a three-bond correlation to a carbon resonance at δ_{C} 163.4, appropriate for an iminium carbon in an iminoquinone moiety. These data showed that the D-ring of tigecycline was converted into an iminium p-quinone moiety from the original aminophenol species. This conversion agrees with the color change from the orange for tigecycline to blue for the iminoquinone derivative of tigecycline since a quinone/iminoquinone chromophore has the longer wavelength absorption than its hydroquinone/aminophenol counterpart.

Other important NMR information supporting the assignment of the structure was as follows: A proton doublet at δ_{H} 4.26 was assigned to H-6 that correlated to a carbon resonance at δ_{C} 72.5 (C-6) from the HSQC experiment. The magnitude of both proton and carbon chemical shifts specified that C-6 was an oxygenated carbon, possibly a secondary alcohol. In the COSY spectrum, H-6 was coupled to a proton signal at δ_{H} 1.96 (H-5a), the latter further correlated to a resonance at δ_{H} 2.55 (H-5). The presence of this spin system was supported by TOCSY spectrum that showed correlations from H-5 to both H-6 and H-5a. The multiplicity-edited HSQC experiment determined that H-5a and H-5 were methine and methylene protons and their attached carbons resonated at δ_{C} 44.9 (C-5a) and δ_{C} 33.5 (C-5), respectively. A coupling constant of 11.3 Hz between H-6 and H-5a indicated that they were in trans orientation on the C-ring, a typical J-coupling value in a 6-membered ring system. The remaining dimethyl amino group on the A-ring was detected as two very broad peaks at δ_{H} 3.25 and δ_{H} 2.55. Both peaks correlated with each other in the TOCSY spectrum but not in the COSY spectrum, indicating that these two signals were exchangeable. This could be explained as protonated (δ_{H} 3.25) and de-protonated (δ_{H} 2.55) versions of the dimethyl amino group. Due to the opening of the B-ring, the A-ring could very likely have rotated around the C-4a/C-5 bond to form a zwitterions. In addition, the A-ring was aromatized into a more stable form as evidenced by no aliphatic methine proton/carbon resonances detected for C-4. Identification of the t-butylamino-acetamide group was straightforward as its NMR data was consistent with that of tigecycline (Table 2). The exchangeable protons were not observed in deuterated water. In addition, due to the stability issue and the limitations of sample concentration, no direct ¹³C NMR spectra were obtained. However, the NMR data together with the LC-MS results clearly supported the compound I.

What is claimed is:

1. A method of detecting in a tigecycline solution the presence of the degradant Compound I having the structure 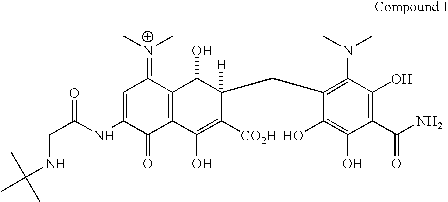
said method comprising:
(a) measuring spectrophotometrically the color indices L*, a* and b* of the solution; and,
(b) calculating a color value by inserting the measured color indices into the discriminator equation,
calculated color value=(k1×L*+k2×a*+k3×b*),
wherein k1 is approximately 19, k2 is approximately 2.59, and k3 is approximately −0.48.

2. The method according to claim 1 further comprising comparing the calculated color value to a selected color value to determine whether the amount of degradant is unacceptable, wherein the amount is unacceptable if the calculated color value is less than the selected color value.

3. The method according to claim 2 wherein the selected color value is approximately 150.

4. A substantially pure compound having the chemical structure [chemical structure]
